(19) United States
(12) Patent Application Publication
(10) Pub. No.: US 2012/0053238 A1
(43) Pub. Date: Mar. 1, 2012

(54) SOLID RETIGABINE IN NON-CRYSTALLINE FORM

(75) Inventors: Jana Paetz, North Rhine-Westphalia (DE); Katrin Rimkus, North Rhine-Westphalia (DE)

(73) Assignee: ratiopharm GmbH, Ulm (DE)

(21) Appl. No.: 13/257,061

(22) PCT Filed: Mar. 17, 2010
(86) PCT No.: PCT/EP2010/001691

§ 371 Date: Nov. 8, 2011

(30) Foreign Application Priority Data
Mar. 17, 2009 (DE) 1020090136118

(51) Int. Cl. A61K 31/27; G01N 25/02; C07C 271/28

(52) U.S. Cl. 514/487; 560/27; 374/25

(57) ABSTRACT

The invention relates to solid retigabine in non-crystalline form together with a surface stabiliser in the form of a stable intermediate. In the intermediate of the invention, retigabine is preferably present in amorphous form or in the form of a solid solution. The invention further relates to processes for the production of retigabine in a solid, non-crystalline form and to pharmaceutical formulations containing solid, non-crystalline retigabine.

The invention relates to solid retigabine in non-crystalline form together with a surface stabiliser in the form of a stable intermediate. In the intermediate of the invention, retigabine is preferably present in amorphous form or in the form of a solid solution. The invention further relates to processes for the production of retigabine in a solid, non-crystalline form and to pharmaceutical formulations containing solid, non-crystalline retigabine.

The IUPAC name of retigabine [INN] is 2-amino-4-(4-fluorobenzylamino)-1-ethoxycarbonyl aminobenzene. The chemical structure of retigabine is shown in formula (1) below:

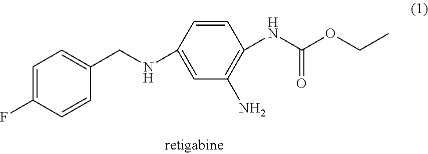

Synthesis pathways for crystalline retigabine and its use as an anti-epileptic agent have been described in EP 0 554 543. The use of retigabine for the treatment of neuropathic pain is also known from WO 01/22953 A2.

Epilepsy is one of the commonest neurological disorders and affects up to about 1% of the population. Whereas a majority of epilepsy patients can be treated with anticonvulsants currently available on the market, about 30% of patients are pharmacoresistant. There is therefore a need to develop new anticonvulsants with innovative mechanisms of action. As a potassium channel opener, retigabine, an anticonvulsant substance, satisfies these criteria. As yet, however, no pharmaceutical dosage forms are known in the art which permit an advantageous, oral administration of retigabine in high doses, especially with modified release, for the treatment of epilepsy.

WO 02/80898 A2 proposes formulating crystalline retigabine in the form of hard gelatine capsules containing 50, 100 and 200 mg active agent. Hard gelatine capsules are often felt by patients to be unpleasant to take. In particular, it is problematic to obtain a high content of active agent (e.g. 70%) in the capsule with this method. It has also become apparent that capsules produced by means of the wet granulation of crystalline retigabine are not ideal with regard to their pharmacokinetic properties.

In addition, retigabine formulations are proposed in WO 01/66081 A2 which were produced by melt granulation at 50 to 60° C., where a matrix composition consisting solely of crystalline retigabine and sucrose fatty acid ester resulted. The use of large amounts of sucrose fatty acid ester is often undesirable, however, because of the emulsifier effect. Furthermore, the formulations proposed merely permit delayed release.

The object of the present invention was therefore to overcome the above-mentioned disadvantages. The intention is to provide the active agent in a form possessing good flowability and thus making it possible for it to be processed not only into capsules, but also to ensure good compression into tablets. It is also the intention to provide the active agent in a form which does not have a tendency to agglomerate. In addition, it is intended to ensure an even distribution of the active agent. It is intended to avoid micronisation of the active agent.

While developing retigabine formulations, the inventors of the present application were also confronted with the fact that crystalline retigabine can exist in different crystalline polymorphous forms. As described in WO 98/31663, these polymorphs are frequently not stable, however, but tend to change into different crystalline, polymorphous forms. The frequently used retigabine form A, for example, can change into form B under the influence of heat. However, the polymorphous forms A, B and C have different solubility profiles.

In a patient, the different solubility profile leads to an undesirable, uneven rise in the concentration of the active agent. It is therefore an object of the present invention to provide stable retigabine intermediates that can be processed into a dosage form which enables as even a rise as possible in the concentration in the patient (even after storage). The aim is largely to avoid both inter-individual and also intra-individual deviations.

The intention is also to provide dosage forms of retigabine which ensure good solubility and bioavailability with good storage stability at the same time.

All the objects mentioned above are supposed to be achieved in particular for a high content of active agent (drug load).

It was unexpectedly possible to solve the problems by converting retigabine, especially crystalline retigabine, into a solid, non-crystalline form, especially a stabilised amorphous form, or into the form of a solid solution.

The subject matter of the invention is therefore retigabine in solid, non-crystalline form, wherein the retigabine is present together with a surface stabiliser. In the context of this application, two possible embodiments of retigabine in solid, non-crystalline form are illustrated from this point of view.

In a first embodiment, the subject matter of the invention is therefore an intermediate containing amorphous retigabine and a surface stabiliser. That intermediate is amorphous retigabine in stabilised form.

In a second embodiment, the subject matter of the invention is an intermediate containing retigabine in the form of a solid solution and a surface stabiliser. In this second embodiment, the surface stabiliser acts as a “matrix material”, in which retigabine is present distributed in a molecularly disperse manner. The intermediate is a solid solution of retigabine in stabilised form.

The subject matter of the invention is also various processes for the production of solid non-crystalline retigabine in the form of the intermediate of the invention.

Finally, the subject matter of the invention comprises pharmaceutical formulations containing the solid, non-crystalline retigabine of the invention or the stabilised retigabine of the invention in the form of the intermediates of the invention.

In the context of this invention, the term “retigabine” comprises 2-amino-4-(4-fluoro-benzylamino)-1-ethoxycarbonyl aminobenzene according to the above formula (1). In addition, the term “retigabine” comprises all the pharmaceutically acceptable salts and solvates thereof.

The salts may be acid addition salts. Examples of suitable salts are hydrochlorides (monohydrochloride, dihydrochloride), carbonates, hydrogen carbonates, acetates, lactates, butyrates, propionates, sulphates, methane sulphonates, citrates, tartrates, nitrates, sulphonates, oxalates and/or succinates. Retigabine is preferably used in the form of the free base.

The first embodiment of the present invention relates to amorphous retigabine. The term “amorphous” is used in the context of this invention to denote the state of solid substances in which the components (atoms, ions or molecules, i.e. in the case of amorphous retigabine the retigabine molecules) do not exhibit any periodic arrangement over a great range (=long-range order). In amorphous substances, the components are usually not arranged in a totally disordered fashion and completely randomly, but are rather distributed in such a way that a certain regularity and similarity to the crystalline state can be observed with regard to the distance from and orientation towards their closest neighbours (=short-range order). Amorphous substances consequently preferably possess a short-range order, but no long-range order. In addition, an amorphous substance, especially amorphous retigabine, usually has an average particle size of more than 300 nm.

In contrast to anisotropic crystals, solid amorphous substances are isotropic. Normally, they do not have a defined melting point, but instead gradually pass over into the liquid state after slowly softening. They can be distinguished from crystalline substances experimentally by means of X-ray diffraction, which does not reveal clearly defined interferences for them, but rather, in most cases, only a few diffuse interferences with small diffraction angles.

In the context of the first embodiment of this invention, the expression “amorphous retigabine” preferably refers to a substance which consists of amorphous retigabine. Alternatively, “amorphous retigabine” may also contain small amounts of crystalline retigabine components, provided that no defined melting point of crystalline retigabine can be detected in DSC. A mixture containing 90 to 99.99% by weight amorphous retigabine and 0.01 to 10% crystalline retigabine is preferred, more preferably 95 to 99.9% by weight amorphous retigabine and 0.1 to 5% crystalline retigabine.

In the context of this first embodiment of the invention, the retigabine of the invention is present in stabilised form, namely in the form of an intermediate containing amorphous retigabine and a surface stabiliser. In particular, the intermediate of the invention consists substantially of amorphous retigabine and surface stabiliser. If—as described below—a crystallisation inhibitor is used in addition, the intermediate of the invention may consist substantially of amorphous retigabine, surface stabiliser and crystallisation inhibitor. The expression “substantially” in this case indicates that small amounts of solvent etc. may optionally also be present.

The second embodiment of the present invention relates to retigabine in the form of a solid solution. The term “solid solution” is to be understood in the context of this invention as meaning that retigabine is distributed in a molecularly disperse manner in a matrix which is present in a solid aggregate state at 25° C.

It is preferable that in this second embodiment, the intermediate of the invention (containing retigabine in the form of a solid solution) contains substantially no crystalline or amorphous retigabine. In particular, the intermediate of the invention contains less than 15% by weight, more preferably less than 5% by weight, of amorphous or crystalline retigabine, based on the total weight of the retigabine present in the intermediate.

It is further preferred that “molecularly disperse” should be understood as meaning that the intermediate of the invention does not contain any retigabine particles with a particle size greater than 300 nm, more preferably greater than 200 nm, especially greater than 100 nm. The particle size is determined in this connection by means of confocal Raman spectroscopy. The measuring system preferably consists of an NTEGRA-Spektra Nanofinder ex NT-MDT.

In the context of this second embodiment of this invention, the solid solution of retigabine of the invention is present in stabilised form, namely in the form of an intermediate containing molecularly disperse retigabine and a surface stabiliser (as a matrix material). In particular, the intermediate of the invention consists substantially of molecularly disperse retigabine and matrix material. If—as described below—a crystallisation inhibitor is used in addition, the intermediate of the invention may consist substantially of molecularly disperse retigabine, surface stabiliser and crystallisation inhibitor. The expression “substantially” in this case indicates that small amounts of solvent etc. may optionally also be present.

Both embodiments of the present invention relate to an intermediate containing a surface stabiliser. The surface stabiliser is generally a substance which is suitable for stabilising retigabine in amorphous form or in the form of a solid solution. The surface stabiliser is preferably a polymer. In addition, the surface stabiliser also includes substances which behave like polymers. Examples of these are fats and waxes. Furthermore, the surface stabiliser also includes solid, non-polymeric compounds which preferably contain polar side groups. Examples of these are sugar alcohols or disaccharides.

A further subject matter of the invention is a method of identifying a pharmaceutical excipient which is suitable as a surface stabiliser for solid, non-crystalline (i.e. amorphous retigabine or for retigabine in the form of a solid solution) and which can hence be used for preparing the intermediate of the invention. The method comprises the steps of:

a) Providing a pharmaceutical excipient which is present in a solid aggregate state at 25° C. For this purpose, it is generally possible to choose the pharmaceutical excipients mentioned in the European Pharmacopoeia.

b) Twice in succession, heating up the solid excipient by means of DSC. In this case, two heating curves are recorded by means of DSC. The curves are usually recorded from 20° C. to no more than 20° C. below the decomposition range of the substance to be tested.

For this purpose a Mettler Toledo DSC 1 apparatus can be used. The work is performed at a heating rate of 1-20° C./min, preferably 5-15° C./min, and at a cooling rate of 5-25° C./min, preferably 10-20° C./min.

c) Selecting the excipient as “suitable” if a glass transition point of 20 to 120° C., preferably 25° C. to 100° C., can be seen in the second DSC heating curve.

Another subject matter of the invention is intermediates containing solid, non-crystalline retigabine (i.e. amorphous retigabine or retigabine in the form of a solid solution) and a pharmaceutical excipient selected by means of the method described above.

The surface stabiliser used for the preparation of the intermediate of the invention is preferably a polymer. The polymer to be used for the preparation of the intermediate preferably has a glass transition temperature (Tg) of more than 20° C. and less than 200° C., more preferably from 30° C. to 150° C., especially from 40° C. to 100° C. By immobilisation, a polymer with a Tg selected accordingly prevents the recrystallisation of the amorphous retigabine or prevents the reversion of the molecular retigabine dispersion into colloids or particles.

The term “glass transition temperature” (Tg) is used to describe the temperature at which amorphous or partially crystalline polymers change from the solid state to the liquid state. In the process, a distinct change in physical parameters, e.g. hardness and elasticity, occurs. Below the Tg, a polymer is usually glassy and hard, whereas above the Tg, it changes into a rubber-like to viscous state. The glass transition temperature is determined in the context of this invention by means of dynamic differential scanning calorimetry (DSC). For this purpose a Mettler Toledo DSC 1 apparatus can be used. The work is performed at a heating rate of 1-20° C./min, preferably 5-15° C./min, and at a cooling rate of 5-25° C./min, preferably 10-20° C./min.

In addition, the polymer which can be used to produce the intermediate preferably has a weight-average or number-average molecular weight of 1,000 to 500,000 g/mol, more preferably 2,000 to 90,000 g/mol. When the polymer used to produce the intermediate is dissolved in water in an amount of 2% by weight, the resulting solution preferably has a viscosity of 0.1 to 8 mPa×s, more preferably 0.5 to 15 mPa×s, especially 1.0 to 8 mPa×s, measured at 25° C. and preferably determined in accordance with Ph. Eur., 6th edition, chapter 2.2.10.

Hydrophilic polymers are preferably used for the preparation of the intermediate. This refers to polymers which possess hydrophilic groups. Examples of suitable hydrophilic groups are hydroxy, alkoxy, acrylate, methacrylate, sulphonate, carboxylate and quaternary ammonium groups.

The intermediate of the invention may, for example, comprise the following hydrophilic polymers as the surface stabiliser: polysaccharides, such as hydroxypropyl methyl cellulose (HPMC), carboxymethyl cellulose (CMC, especially sodium and calcium salts), ethyl cellulose, methyl cellulose, hydroxyethyl cellulose, ethyl hydroxyethyl cellulose, hydroxypropyl cellulose (HPC) , e.g. L-HPC (low substituted hydroxypropyl cellulose); microcrystalline cellulose, polyvinyl pyrrolidone, polyvinyl acetate (PVAC), polyvinyl alcohol (PVA), polymers of acrylic acid and their salts, polyacrylamide, polymethacrylates, vinyl pyrrolidone/vinyl acetate copolymers (such as Kollidon® VA64, BASF), polyalkylene glycols, such as polypropylene glycol or preferably polyethylene glycol, co-block polymers of polyethylene glycols, especially co-block polymers of polyethylene glycol and polypropylene glycol (Pluronic®, BASF) and mixtures of the polymers mentioned.

It is preferable that the polymers used as surface stabilisers should exhibit substantially no emulsifying effect. This means that the surface stabiliser used should preferably not contain any combination of hydrophilic and hydrophobic groups (especially hydrophobic fatty acid groups). In particular, the surface stabiliser is not a sucrose fatty acid ester. In addition, it is preferable for the intermediate of the invention not to contain any polymers that have a weight-average molecular weight of more than 150,000 g/mol. It may happen that polymers of this kind have an undesirable influence on the dissolution characteristics.

Substances particularly preferably used as adhesion promoters are polyvinyl pyrrolidone, preferably with a weight-average molecular weight of 10,000 to 60,000 g/mol, especially 12,000 to 40,000 g/mol, a copolymer of vinyl pyrrolidone and vinyl acetate, especially with a weight-average molecular weight of 40,000 to 70,000 g/mol and/or polyethylene glycol, especially with a weight-average molecular weight of 2,000 to 10,000 g/mol, and HPMC, especially with a weight-average molecular weight of 20,000 to 90,000 g/mol and/or preferably a content of methyl groups of 10 to 35% and a content of hydroxy groups of 1 to 35%. In addition microcrystalline cellulose can preferably be used, especially one with a specific surface area of 0.7-1.4 m²/g. The specific surface area is determined by means of the gas adsorption method according to Brunauer, Emmet and Teller. Their weight-average molecular weight is usually determined by means of gel permeation chromatography. The copolymer of vinyl pyrrolidone and vinyl acetate preferably has the following structural unit.

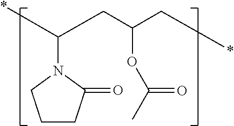

For the surface stabiliser, it is also particularly preferable to use co-block polymers of polyethylene glycol and polypropylene glycol, i.e. polyoxyethylene polyoxypropylene block polymers. These preferably have a weight-average molecular weight of 1,000 to 20,000 g/mol, more preferably 1,500 to 12,500 g/mol, especially 5,000 to 10,000 g/mol. These block polymers are preferably obtainable by condensation of propylene oxide with propylene glycol and subsequent condensation of the polymer formed with ethylene oxide. This means that the ethylene oxide content is preferably present as an “endblock”. The block polymers preferably have a weight ratio of propylene oxide to ethylene oxide of 50:50 to 95:5, more preferably 70:30 to 90:10. The block polymers preferably have a viscosity at 25° C. of 200 to 2,000 mPas, more preferably 500 to 1,500 mPas, especially 800 to 1,200 mPas.

In addition, the surface stabiliser also includes solid, non-polymeric compounds, which preferably contain polar side groups. Examples of these are sugar alcohols or disaccharides. Examples of suitable sugar alcohols and/or disaccharides are mannitol, sorbitol, xylitol, isomalt, glucose, fructose, maltose and mixtures thereof. The term “sugar alcohols” in this context also includes monosaccharides. In particular, isomalt and sorbitol are used as the surface stabiliser.

In the context of this invention, no sucrose fatty acid esters are used as surface stabilisers.

In a preferred embodiment, the intermediate of the invention contains solid, non-crystalline retigabine (i.e. amorphous retigabine or retigabine in the form of a solid solution) and surface stabiliser, wherein the weight ratio of solid, non-crystalline retigabine to surface stabiliser is 10:1 to 1:10, more preferably 5:1 to 1:3, even more preferably 3:1 to 1:2, especially 2:1 to 1:1.5.

It is preferable that that type and quantity of surface stabiliser should be selected such that the resulting intermediate has a glass transition temperature (Tg) of more than 20° C., preferably >30° C. In addition, the resulting intermediate has a Tg of less than 180° C., more preferably less than 120° C.

It is preferable that type and quantity of the polymer should be selected such that the resulting intermediate is storage-stable. “Storage-stable” means that in the intermediate of the invention, after storage for 3 years at 25° C. and 50% relative humidity, the proportion of crystalline retigabine—based on the total amount of retigabine—is no more than 60% by weight, preferably no more than 30% by weight, more preferably no more than 15% by weight, in particular no more than 5% by weight.

It is advantageous for the surface stabiliser or the matrix material to be used in particulate form, wherein the volume-average particle size (D50) is less than 500 μm, preferably 5 to 250 μm.

In a preferred embodiment, in addition to solid, non-crystalline retigabine (i.e. in addition to amorphous retigabine or retigabine in the form of a solid solution) and surface stabiliser, the intermediates of the invention also contain a crystallisation inhibitor based on an inorganic salt, an organic acid or a polymer with a weight-average molecular weight (Mw) of more than 500,000 g/mol. These polymers which are suitable as crystallisation inhibitors are also referred to in the context of this invention as “high-viscosity polymers”. Their weight-average molecular weight is usually less than 5,000,000 g/mol. A preferred high-viscosity polymer is povidone.

The crystallisation inhibitor is preferably ammonium chloride, citric acid, or Povidone K 90 (in accordance with Ph. Eur. 6.0).

The crystallisation inhibitor can generally be used in an amount of 1 to 30% by weight, preferably 2 to 25% by weight, more preferably 5 to 20% by weight, based on the total weight of the intermediate.

The intermediates of the invention are obtainable by a variety of preparation methods. Depending on the preparation method, the intermediates are obtained in different particle sizes. Normally, the intermediates of the invention are present in particulate form and have an average particle diameter (D50) of 1 to 750 μm, depending on the preparation method.

The expression “average particle diameter” relates in the context of this invention to the D50 value of the volume-average particle diameter determined by means of laser diffractometry. In particular, a Malvern Instruments Mastersizer 2000 was used to determine the diameter (wet measurement with ultrasound for 60 sec., 2,000 rpm, the evaluation being performed using the Fraunhofer model), and preferably using a dispersant in which the substance to be measured does not dissolve at 20° C.).

The average particle diameter, which is also referred to as the D50 value of the integral volume distribution, is defined in the context of this invention as the particle diameter at which 50% by volume of the particles have a smaller diameter than the diameter which corresponds to the D50 value. Similarly, 50% by volume of the particles then have a larger diameter than the D50 value.

Another subject matter of the invention is processes for preparing the intermediate of the invention. In the following, five preferred embodiments of such processes will be explained. Processes (1) to (3) here are suitable for the production of both amorphous retigabine (=first embodiment of the intermediate of the invention) and also retigabine in the form of a solid solution (second embodiment of the intermediate of the invention). Processes (4) and (5) are preferably used to produce amorphous retigabine. In particular, process (3) is used for the production of amorphous retigabine and/or retigabine or in the form of a solid solution.

In a first preferred method, the invention relates to a “pellet-layering process”, i.e. a process for preparing an intermediate of the invention, comprising the steps of
(a1) dissolving the retigabine and the surface stabiliser in a solvent or mixture of solvents, and
(b1) spraying the solution from step (al) onto a substrate core.

In step (a1), retigabine and the surface stabiliser described above are dissolved, preferably completely dissolved, in a solvent or mixture of solvents. It is preferable to use crystalline retigabine for this purpose. In addition, it is preferable for retigabine to be used in the form of one of the acid addition salts described above; retigabine dihydrochloride, for example, can advantageously be used.

Suitable solvents are, for example, water, alcohol (e.g. methanol, ethanol, isopropanol), dimethyl sulphoxide (DMSO), acetone, butanol, ethyl acetate, heptane, pentanol or mixtures thereof. Preferably, a mixture of water and ethanol is used.

Suitable surface stabilisers in this first method are in particular modified celluloses, such as HPMC (preferably with a weight-average molecular weight of 20,000 to 90,000 g/mol), sugar alcohols, such as isomalt and sorbitol, and polyethylene glycol, especially polyethylene glycol with a molecular weight of 2,000 to 10,000 g/mol.

If the intermediate to be prepared is additionally intended to contain a crystallisation inhibitor based on an inorganic salt or an organic acid, or a highly viscous polymer, this can likewise be added in step (a1). Reference is made to the above statements with regard to the type and amount of the crystallisation inhibitor.

In step (b1), the solution from step (a1) is sprayed onto a substrate core. Suitable substrate cores are particles consisting of pharmaceutically acceptable excipients, especially “neutral pellets”. The pellets preferably used are those which are available under the trade name Cellets® and which contain a mixture of lactose and microcrystalline cellulose, or sugar spheres, which are a mixture of starch and sugar.

Step (b1) is preferably performed in a fluidised bed dryer, such as a Glatt GPCG 3 (Glatt GmbH, Germany). Work is preferably performed with air inlet temperatures of 50 to 100° C., preferably von 60 to 80° C., with product temperatures of 25 to 50° C., preferably 30 to 40° C. and with a spray pressure of 0.9 to 2.5 bar, preferably 1 to 1.5 bar.

Depending on the choice of starting materials in step (a1) and the process parameters in step (b1), the resulting intermediate may contain retigabine in amorphous form or in the form of a solid solution.

It is difficult to make a general statement, because these steps are heavily dependent on the molecule. First of all, it is necessary to characterise the molecule per se more specifically in order to be able to draw conclusions afterwards.

The process conditions in this first method are preferably selected such that the resulting intermediate particles have a volume-average particle diameter (D50) of 50 to 800 μm, more preferably 150 to 650 μm, especially 200 to 600 μm.

In a second preferred method, the invention relates to a spray-drying process for preparing the intermediate of the invention, comprising the steps of
(a2) dissolving retigabine and the surface stabiliser in a solvent or mixture of solvents, and
(b2) spray-drying the solution from step (a2).

In step (a2), retigabine and the matrix material described above are dissolved, preferably completely dissolved, in a solvent or mixture of solvents. It is preferable to use crystalline retigabine. In addition, it is preferable for retigabine to be used in the form of one of the acid addition salts described above; retigabine dihydrochloride, for example, can advantageously be used.

Suitable solvents are, for example, water, alcohol (e.g. methanol, ethanol, isopropanol), dimethyl sulphoxide (DMSO), acetone, butanol, ethyl acetate, heptane, pentanol or mixtures thereof. Preferably, an ethanol/water mixture is used.

Suitable surface stabilisers in this first method are in particular modified celluloses, such as HPMC (preferably with a weight-average molecular weight of 20,000 to 90,000 g/mol), polyvinyl pyrrolidone and copolymers thereof (preferably with a weight-average molecular weight of 20,000 to 70,000 g/mol) and sugar alcohols, such as isomalt and sorbitol.

If the intermediate to be prepared is additionally intended to contain a crystallisation inhibitor based on an inorganic salt or an organic acid, or a highly viscous polymer, this can likewise be added in step (a2). Reference is made to the above statements with regard to the type and amount of the crystallisation inhibitor.

In the subsequent step (b2), the solution from step (a2) is spray-dried. The spray-drying is usually carried out in a spray tower. As an example, a Büchi B-191 is suitable (Büchi Labortechnik GmbH, Germany). Preferably an inlet temperature of 100° C. to 150° C. is chosen. The amount of air is, for example, 500 to 700 litres/hour, and the aspirator preferably runs at 80 to 100%.

Depending on the choice of starting materials in step (a2) and the process parameters in step (b2), the resulting intermediate may contain retigabine in amorphous form or in the form of a solid solution.

The process conditions in this second method are preferably selected such that the resulting intermediate particles have a volume-average particle diameter (D50) of 1 to 250 μm, more preferably 5 to 150 μm, especially 10 to 100 μm.

In a third preferred method, the invention relates to a melt-processing method, preferably a melt-extrusion process, i.e. a method of preparing the intermediate of the invention, comprising the steps of
(a3) mixing retigabine and surface stabiliser, and
(b3) melt-processing, preferably melt-extruding, the mixture, the melt-processing conditions, preferably extrusion conditions, being selected such that there is a transition from crystalline to non-crystalline retigabine.

In step (a3), crystalline retigabine is mixed with the surface stabiliser, preferably in a mixer. In this version of the method of the invention, a matrix material (i.e. a surface stabiliser) is preferably used in polymeric form is used. In addition, retigabine is preferably used in the form of the free base.

Suitable polymeric surface stabilisers in this third method are especially polyvinyl pyrrolidone and vinyl pyrrolidone/vinyl acetate copolymers, and also polyvinyl alcohols, methacrylates, PEG and HPMC. The weight-average molecular weight of the polymers used is usually 4,000 to 80,000 g/mol, preferably 6,000 to 50,000 g/mol.

If the intermediate to be prepared is additionally intended to contain a crystallisation inhibitor based on an inorganic salt or an organic acid, or a highly viscous polymer, this can likewise be added in step (a3). Reference is made to the above statements with regard to the type and amount of the crystallisation inhibitor.

In step (b3), the mixture is melt-processed, preferably extruded. In the course of the melt-processing (b3), retigabine is processed with the—preferably polymeric, especially thermoplastic—surface stabiliser in such a way that retigabine is embedded in the surface stabiliser in non-crystalline form. The melt processing can preferably be carried out as melt granulation or melt extrusion.

The mixture from step (a3) is conventionally processed in the extruder into a homogeneous melt. The extrusion conditions are preferably selected such that there is a transition from crystalline to non-crystalline retigabine.

The extruders used may be conventional melt extruders, such as a Leistritz® Micro 18. The melt-processing temperature or extrusion temperature depends on the nature of the matrix material. It usually lies between 80 and 250° C., preferably between 100 and 180° C., especially between 105 and 150° C. The extrusion is preferably carried out at an outlet pressure of 10 bar to 100 bar, more preferably at 20 to 80 bar.

The cooled melt is usually comminuted by a rasp screen (e.g. Comill® U5) and in this way accordingly reduced to a uniform particle size.

Depending on the choice of starting materials in step (a3) and the process parameters in step (b3), the resulting intermediate may contain retigabine in amorphous form or in the form of a solid solution. In particular, it has proven suitable for the extruder to be equipped with a kneader unit if retigabine is to be obtained in the form of a solid solution. The kneader unit should be designed such that intensive blending is ensured, so that a solution of retigabine in the surface stabiliser is ensured.

The process conditions in this third method are preferably selected such that the resulting intermediate particles have a volume-average particle diameter (D50) of 150 to 1,000 μm, more preferably a D50 of 250 to 600 μm.

Instead of granulating the extruded material, “direct injection moulding” may also be performed. In this case, the method of the invention includes the step of
(c3) injection moulding the extruded material into moulds for pharmaceutical dosage forms.

Examples are moulds for tablets.

Melt-processing, preferably melt-extrusion, is the particularly preferred process for the production of non-crystalline retigabine.

In a fourth preferred method, the invention relates to a freeze-drying process, i.e. a process for preparing the intermediate of the invention, comprising the steps of
(a4) dissolving the retigabine, preferably the crystalline retigabine and the surface stabiliser, in a solvent or mixture of solvents, and
(b4) freeze-drying the solution from step (a4).

In step (a4), retigabine, preferably crystalline retigabine and the surface stabiliser described above, is dissolved, preferably completely dissolved, in a solvent or mixture of solvents. In addition, it is preferable for retigabine to be used in the form of one of the acid addition salts described above; retigabine dihydrochloride, for example, can advantageously be used.

Suitable solvents are, for example, water, alcohol (e.g. methanol, ethanol, isopropanol), dimethyl sulphoxide (DMSO), acetone, butanol, ethyl acetate, heptane, pentanol or mixtures thereof. Preferably, a mixture of water and ethanol is used.

Suitable surface stabilisers in this method are especially modified celluloses such as HPMC (preferably with a weight-average molecular weight of 20,000 to 90,000 g/mol) and sugar alcohols such as isomalt, mannitol and sorbitol.

If the intermediate to be prepared is additionally intended to contain a crystallisation inhibitor based on an inorganic salt or an organic acid, or a highly viscous polymer, this can likewise be added in step (a4). Reference is made to the above statements with regard to the type and amount of the crystallisation inhibitor.

The solution from step (a4) is cooled to about 10 to 50° C. below freezing point (i.e. it is frozen). Then the solvent is removed by sublimation. This is preferably done when the conductivity of the solution is less than 2%. The sublimation temperature is preferably determined by the point of intersection of the product temperature and Rx −10° C. Sublimation is preferably effected at a pressure of less than 0.1 mbar.

After completion of the sublimation, the lyophilised intermediate is heated to room temperature.

The process conditions in this fourth method are preferably selected such that the resulting intermediate particles have a volume-average particle diameter (D50) of 1 to 250 μm, more preferably 3 to 150 μm, especially 5 to 100 μm.

In a fifth preferred method, the invention relates to a milling process, i.e. a process for preparing the intermediate of the invention, comprising the steps of
(a5) mixing retigabine, preferably crystalline retigabine, and surface stabiliser, and
(b5) milling the mixture from step (a5), the milling conditions preferably being selected such that there is a transition from crystalline to amorphous retigabine.

Crystalline retigabine and surface stabiliser are preferably mixed in step (a5). The mixture is milled in step (b5). The mixing may take place before or even during the milling, i.e. steps (a5) and (b5) may be performed simultaneously.

If the intermediate to be prepared is additionally intended to contain a crystallisation inhibitor based on an inorganic salt or an organic acid, this can likewise be added in step (a5) or (b5). Reference is made to the above statements with regard to the type and amount of the crystallisation inhibitor.

The milling conditions are preferably selected such that there is a transition from crystalline to amorphous retigabine.

The milling is generally performed in conventional milling apparatuses, preferably in a ball mill, such as a Retsch PM 100.

The milling time is usually 10 minutes to 10 hours, preferably 30 minutes to 8 hours, more preferably 2 hours to 6 hours.

Suitable surface stabilisers in this fifth method are in particular polyvinyl pyrrolidone, modified celluloses, such as HPMC, sugar alcohols, such as isomalt and sorbitol, and polyethylene glycol, especially polyethylene glycol with a molecular weight of 2,000 to 10,000 g/mol.

The process conditions in this fourth method are preferably selected such that the resulting intermediate particles have a volume-average particle diameter (D₅₀) of 1 to 350 μm, more preferably 10 to 150 μm, especially 20 to 120 μm.

The intermediate of the invention (i.e. the stabilised non-crystalline retigabine of the invention) is usually employed to prepare a pharmaceutical formulation.

One subject matter of the invention is therefore a pharmaceutical formulation containing intermediate of the invention and pharmaceutical excipients.

These are the excipients with which the person skilled in the art is familiar, such as those which are described in the European Pharmacopoeia.

Examples of excipients used are disintegrants, anti-stick agents, emulsifiers, pseudoemulsifiers, fillers, additives to improve the powder flowability, glidants, wetting agents, gel-forming agents and/or lubricants. Where appropriate, further excipients can also be used.

The ratio of active agent to excipients is preferably selected such that the resulting formulations contain

40 to 90% by weight, more preferably 55 to 85% by weight, especially 60 to 80% by weight non-crystalline retigabine and

10 to 60% by weight, more preferably 15 to 45% by weight, especially 20 to 40% by weight pharmaceutically acceptable excipients.

In these ratios specified, the amount of surface stabiliser used to prepare the intermediate of the invention is counted as an excipient. This means that the amount of active agent refers to the amount of non-crystalline retigabine contained in the formulation.

It has been shown that the intermediates of the invention are suitable for serving both as a basis for a dosage form with immediate release (or “IR” for short) and also with modified release (or “MR” for short).

In a preferred embodiment for an IR formulation, a relatively large amount of disintegrant is used. In that preferred embodiment, the pharmaceutical formulation of the invention therefore contains

1 to 30% by weight, more preferably 3 to 15% by weight, especially 5 to 12% by weight disintegrants, based on the total weight the formulation.

“Disintegrants” is the term generally used for substances which accelerate the disintegration of a dosage form, especially a tablet, after it is placed in water. Suitable disintegrants are, for example, organic disintegrants such as carrageenan, croscarmellose and crospovidone. Alkaline disintegrants can likewise be used. The term “alkaline disintegrants” means disintegrants which, when dissolved in water, produce a pH level of more than 7.0.

More preferably, inorganic alkaline disintegrants are used, especially salts of alkali and alkaline earth metals. Preferred examples here are sodium, potassium, magnesium and calcium. As anions, carbonate, hydrogen carbonate, phosphate, hydrogen phosphate and dihydrogen phosphate are preferred. Examples are sodium hydrogen carbonate, sodium hydrogen phosphate, calcium hydrogen carbonate and the like.

Sodium hydrogen carbonate is particularly preferably used as a disintegrant, especially in the above-mentioned amounts.

In a preferred embodiment for an MR formulation, a relatively small amount of disintegrant is used. In that preferred embodiment, the pharmaceutical formulation of the invention therefore contains

0.1 to 10% by weight, more preferably 0.5 to 8% by weight, especially 1 to 5% by weight disintegrants, based on the total weight the formulation.

In the case of the MR formulation croscarmellose or crospovidone is preferred as the disintegrant.

In addition the conventional retardation techniques can be used for the MR formulation.

Furthermore, the pharmaceutical formulation (both for IR and for MR) preferably contains one or more of the excipients mentioned in the European Pharmacopoeia. These will be explained in more detail below.

The formulation of the invention preferably contains fillers. “Fillers” are generally understood to mean substances which serve to form the body of the tablet in the case of tablets with small amounts of active agent (e.g. less than 70% by weight). This means that fillers “dilute” the active agents in order to produce an adequate tablet-compression mixture. The normal purpose of fillers, therefore, is to obtain a suitable tablet size.

Examples of preferred fillers are starch, starch derivatives, treated starch, talcum, calcium phosphate, sucrose, calcium carbonate, magnesium carbonate, magnesium oxide, maltodextrin, calcium sulphate, dextrates, dextrin, dextrose, hydrogenated vegetable oil, kaolin, sodium chloride, and/or potassium chloride. Prosolv® (Rettenmaier & Söhne, Germany) can likewise be used.

Fillers are normally used in an amount of 0 to 40% by weight, preferably 1 to 25% by weight, based on the total weight of the formulation.

One example of an additive to improve the powder flowability is disperse silicon dioxide, e.g. known under the trade name Aerosil®.

Additives to improve the powder flowability are usually employed in an amount of 0.1 to 3% by weight, based on the total weight of the formulation.

In addition, lubricants may be used. Lubricants are generally used in order to reduce sliding friction. In particular, the intention is to reduce the sliding friction found during tablet pressing between the punches moving up and down in the die and the die wall, on the one hand, and between the edge of the tablet and the die wall, on the other hand. Suitable lubricants are, for example, stearic acid, adipic acid, sodium stearyl fumarate and/or magnesium stearate.

Lubricants are normally used in an amount of 0.1 to 5% by weight, preferably 0.5 to 3% by weight, based on the total weight of the formulation.

It lies in the nature of pharmaceutical excipients that they sometimes perform more than one function in a pharmaceutical formulation. In the context of this invention, in order to provide an unambiguous delimitation, the fiction will therefore preferably apply that a substance which is used as a particular excipient is not simultaneously also used as a further pharmaceutical excipient.

The pharmaceutical formulation of the invention is preferably pressed into tablets. In the state of the art, wet granulation is proposed for this purpose (see WO 02/080898).

It has, however, become apparent that the properties of the resulting tablets can be improved if wet granulation is avoided.

The intermediates of the invention are therefore compressed into tablets by means of direct compression or are subjected to dry granulation before being compressed into tablets. Intermediates with a bulk density of less than 0.5 g/ml are preferably processed by dry granulation.

Direct compression is especially preferred if the intermediate is prepared by means of melt extrusion (process steps (a3) and (b3) or pellet layering (process steps (a1) and (b1)). Dry granulation is particularly preferable if the intermediate is prepared by means of spray drying (process steps (a2) and (b2)), freeze drying (process steps (a4) and (b4)) or milling (process steps (a5) and (b5)).

A further aspect of the present invention therefore relates to a dry-granulation process comprising the steps of
(I) preparing the intermediate of the invention and one or more pharmaceutical excipients (especially those described above);
(II) compacting it into a slug; and
(III) granulating or comminuting the slug.

In step (I), the intermediate of the invention and excipients are preferably mixed. The mixing can be performed in conventional mixers. Alternatively, it is possible that the retigabine intermediate is initially only mixed with part of the excipients (e.g. 50 to 95%) before compacting (II), and that the remaining part of the excipients is added after the granulation step (III). In the case of multiple compacting, the excipients should preferably be mixed in before the first compacting step, between multiple compacting steps or after the last granulation step.

In step (II) of the process of the invention, the mixture from step (I) is compacted into a slug. It is preferable here that it should be dry compacting, i.e. the compacting is preferably performed in the absence of solvents, especially in the absence of organic solvents.

The compacting conditions are usually selected such that the intermediate of the invention is present in the form of a slug of compacted material, the density of the intermediate being 0.8 to 1.3 g/cm³, preferably 0.9 to 1.20 g/cm³, especially 1.01 to 1.15 g/cm³.

The term “density” here preferably relates to the “pure density” (i.e. not to the bulk density or tapped density). The pure density can be determined with a gas pycnometer. The gas pycnometer is preferably a helium pycnometer; in particular, the AccuPyc 1340 helium pycnometer from the manufacturer Micromeritics, Germany, is used.

The compacting is preferably carried out in a roll granulator.

The rolling force is preferably 5 to 70 kN/cm, preferably 10 to 60 kN/cm, more preferably 15 to 50 kN/cm.

The gap width of the roll granulator is, for example, 0.8 to 5 mm, preferably 1 to 4 mm, more preferably 1.5 to 3 mm, especially 1.8 to 2.8 mm.

In step (III) of the process, the slug is granulated. The granulation can be performed with methods known in the state of the art.

In a preferred embodiment, the granulation conditions are selected such that the resulting particles (granules) have a volume-average particle size ((D₅₀) value) of 50 to 800 μm, more preferably 100 to 750 μm, even more preferably 150 to 500 μm, especially 200 to 450 μm.

In a preferred embodiment, the granulation is performed in a screen mill. In this case, the mesh width of the screen insert is usually 0.1 to 5 mm, preferably 0.5 to 3 mm, more preferably 0.75 to 2 mm, especially 0.8 to 1.8 mm.

The granules resulting from step (III) can be further processed into pharmaceutical dosage forms. For this purpose, the granules are filled into sachets or capsules, for example. The granules resulting from step (III) are preferably compressed into tablets (=step IV).

In step (IV) of the process, the granules obtained in step (III) are pressed into tablets, i.e. the step involves compression into tablets. Compression can be performed with tableting machines known in the state of the art.

In step (IV) of the process, pharmaceutical excipients may optionally be added to the granules from step (III).

The amounts of excipients added in step (IV) usually depend on the type of tablet to be produced and the amount of excipients which were already added in steps (I) or (II).

In the case of direct compression, only steps (I) and (IV) of the method described above are performed.

The tableting conditions are preferably selected such that the resulting tablets have a ratio of tablet height to weight of 0.005 to 0.3 mm/mg, particularly preferably 0.05 to 0.2 mm/mg.

The process of the invention is preferably performed such that the tablet of the invention contains retigabine in an amount of more than 200 mg to 1,000 mg, more preferably 250 mg to 900 mg, especially 300 mg to 600 mg. One subject matter of the invention is thus tablets containing 300 mg, 400 mg, 450 mg, 600 mg or 900 mg retigabine.

In addition, the resulting tablets preferably have a hardness of 50 to 300 N, particularly preferably 80 to 250 N, especially 100 to 220 N. The hardness is determined in accordance with Ph. Eur. 6.0, section 2.9.8.

Also, the resulting tablets preferably have a friability of less than 3%, particularly preferably less than 2%, especially less than 1%. The friability is determined in accordance with Ph. Eur. 6.0, section 2.9.7.

Finally, the tablets of the invention usually have a “content uniformity” of 95 to 105% of the average content, preferably 98 to 102%, especially 99 to 101%. (This means that all the tablets have a content of active agent of between 95 and 105%, preferably between 98 and 102%, especially between 99 and 101% of the average content of active agent.) The “content uniformity” is determined in accordance with Ph. Eur. 6.0, section 2.9.6.

In the case of an IR formulation, the release profile of the tablets of the invention after 10 minutes according to the USP method usually indicates a content released of at least 30%, preferably at least 60%, especially at least 90%.

In the case of an MR formulation, the release profile of the tablets of the invention after 60 minutes according to the USP method usually indicates a content released of 10%, preferably 20%, especially 30%.

The above details regarding hardness, friability, content uniformity and release profile preferably relate here to the non-film-coated tablet for an IR formulation. For a modified-release tablet, the release profile relates to the total formulation.

The tablets produced by the process of the invention may be tablets which can be swallowed unchewed (non-film-coated or preferably film-coated). They may likewise be chewable tablets or dispersible tablets. “Dispersible tablet” here means a tablet to be used for producing an aqueous suspension for swallowing.

In the case of tablets which are swallowed unchewed, it is preferable that they be coated with a film layer. For this purpose, the methods of film-coating tablets which are standard in the state of the art can be employed. The above-mentioned ratios of active agent to excipient, however, relate to the uncoated tablet.

For film-coating, macromolecular substances are preferably used, such as modified celluloses, polymethacrylates, polyvinyl pyrrolidone, polyvinyl acetate phthalate, zein and/or shellack or natural gum, such as carrageenan.

The thickness of the coating is preferably 1 to 100 μm, especially 5 to 75 μm.

The invention will now be explained with reference to the following examples.

### EXAMPLES

In Examples 1, 3a, 3b, 4a, 4b, 4c, retigabine is preferably used in the form of retigabine dihydrochloride, the amount specified referring to the amount of retigabine in the form of the free base. This means that the statement of 300 g retigabine corresponds to about 372 g retigabine dihydrochloride.

### Example 1

### Preparation of the Intermediate Containing Amorphous Retigabine by Lyophilisation

The following batch for 10 dosage forms was produced.

4 g retigabine were dissolved in water/ethanol together with 3 g mannitol. That solution was reduced in temperature to −55° C. and frozen. Once the conductivity had reached less than 2%, the frozen mixture, at a temperature determined by the point of intersection between the product temperature and Rx −10° C. was dried at a pressure of less than 0.1 mbar, or the solvent was removed by sublimation.

After drying, the lyophilised material was heated to room temperature (20-25° C.).

It was possible to carry out the further processing in accordance with Examples 5 to 8.

### Example 2

### Preparation of the Intermediate Containing Retigabine in the Form of a Solid Solution by Melt-Extrusion

The following batch for 1,000 dosage forms was produced.

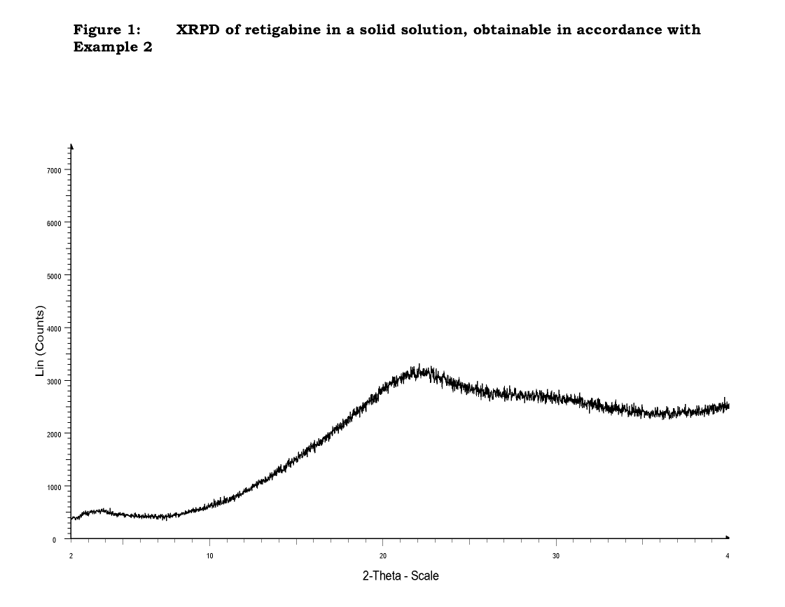

400 g retigabine (preferably in crystalline, polymorphous form A, characterised in accordance with EP 0 956 281 B1) were extruded in a Leistritz micro 18 melt extruder together with 600 g Povidon® VA64 and with a temperature cascade of 90-180° C. The twin-screw extruder was equipped with various screw elements. A kneading unit was installed in order to ensure the necessary thorough mixing and dissolution of the retigabine in the polymer (surface stabiliser). The strands of extruded material were cooled. FIG. 1 shows an XRPD of the resulting intermediate. It can be seen from this that the intermediate of the invention no longer contains any crystalline retigabine.

Further processing was performed after screening on a Comil® US (1.00 mm) in accordance with Examples 5 to 8.

### Example 3a

### Preparation of the Intermediate Containing Amorphous Retigabine by Pellet Layering

The following batch for 100 dosage forms was produced.

40 g retigabine were dissolved in water/ethanol together with 10 g Povidon® VA 64. The solution was applied to 100 g Cellets® in a Glatt® GPC3 fluidised-bed apparatus.

During the process, the air inlet temperature was approx. 60-80° C., the product temperature 32-40° C. and the spray pressure approx. 1-1.5 bar.

It was possible to carry out the further processing in accordance with Examples 5 to 8.

### Example 3b

### Preparation of the Intermediate Containing Retigabine in the Form of a Solid Solution by Pellet Layering

The pellet layering was carried out as described in Example 3a, the following batch being used:

60 g retigabine

20 g sorbitol

It was possible to carry out the further processing in accordance with Examples 5 to 8.

### Example 4a

### Preparation of the Intermediate by Spray-Drying

The following batch for 10 dosage forms was produced.

4 g retigabine were dissolved in water/ethanol together with 4 g HPMC and 0.5 g citric acid and spray-dried on a Büchi TYP B 191 spray tower The following parameters were maintained in the process:

temperature 130° C., spray rate 5-20%, aspirator power 35-90%, flow control 300-700 l/h

The spray-dried material underwent a final drying stage for 24 h at 30° C. in a tray drying cabinet.

It was possible to carry out the further processing in accordance with Examples 5 to 8.

### Example 4b

### Preparation of the Intermediate by Spray-Drying

The spray-drying was carried out as described in Example 4a, the following batch being used:

4 g retigabine

4 g microcrystalline cellulose, a surface stabiliser for the purposes of our invention 1 g Povidon® 25

### Example 4c

### Preparation of the Intermediate by Spray-Drying

The spray-drying was carried out as described in Example 4a, the following batch being used:

4 g retigabine

3 g Povidon® VA 64

It was possible to carry out the further processing in accordance with Examples 5 to 8.

### Example 5

### Production of Tablets by Means of Dry Granulation

In order to produce tablets, the following formulation was used.

| | |
|---|---|
| 1. Intermediate according to Example 2 | 1,500 mg |
| 2. Prosolv ® 90 | 200 mg |
| 3. Talcum | 10 mg |
| 4. Magnesium stearate | 15 mg |
| 5. Aerosil | 30 mg |

Ingredients 1 and 2 were pre-mixed for 5 min in a free-fall mixer (Turbula TB 10). That mixture was compacted with 70% of ingredients 3, 4 and 5 using a roll compactor and screened with a mesh width of 1.25 mm. The compacted material was mixed with the remaining substances and pressed into tablets.

### Example 6

### Production of Tablets by Means of Dry Granulation

In order to produce tablets, the following formulation was used.

| | |
|---|---|
| 1. Intermediate according to Example 2 | 1,500 mg |
| 2. Prosolv ® 90 | 120 mg |
| 3. Talcum | 10 mg |
| 4. Magnesium stearate | 15 mg |
| 5. Aerosil ® | 30 mg |
| 6. Crospovidone | 80 mg |

Ingredients 1, 2 and 6 were pre-mixed for 5 min in a free-fall mixer (Turbula TB 10). This mixture was compacted with 70% of ingredients 3, 4 and 5 using a roll compactor and screened with a mesh width of 1.25 mm. The compacted material was mixed with the remaining substances and pressed into tablets.

### Example 7

### Production of Tablets by Means of Direct Compression

In order to produce tablets, the following formulation was used.

| | |
|---|---|
| 1. Intermediate according to Example 2 | 1,000 mg |
| 2. Calcium hydrogen phosphate | 200 mg |
| 3. Magnesium stearate | 20 mg |
| 4. Aerosil ® | 30 mg |

The intermediate from Example 2 was mixed with calcium hydrogen phosphate for 10 minutes in a free-fall mixer (Turbula T 10B) and screened (1.0 mm), after which the remaining two excipients were added. The finished mixture was compressed on an EK0-type eccentric press (Korsch).

### Example 8

### Production of Tablets by Means of Direct Compression

In order to produce tablets, the following formulation was used.

| | |
|---|---|
| 1. Intermediate according to Example 2 | 1,000 mg |
| 2. Calcium hydrogen phosphate | 120 mg |
| 3. Magnesium stearate | 20 mg |
| 4. Aerosil ® | 30 mg |
| 5. Crospovidone | 80 mg |

The intermediate from Example 2 was mixed with calcium hydrogen phosphate and crospovidone for 10 minutes in a free-fall mixer (Turbula T 10B) and screened (1.0 mm), after which the remaining two excipients were added. The finished mixture was compressed on an EK0-type eccentric press (Korsch).

1. An intermediate comprising retigabine in a solid, non-crystalline form and a surface stabiliser.

2. The intermediate as claimed in claim 1, comprising retigabine in the form of a solid solution and a surface stabiliser.

3. The intermediate as claimed in claim 1, comprising retigabine in an amorphous form and a surface stabiliser.

4. The intermediate as claimed in claim 1, characterised in that the surface stabiliser is a polymer.

5. The intermediate as claimed in claim 1, characterised in that the surface stabiliser is polyvinyl pyrrolidone or a copolymer of vinyl pyrrolidone and vinyl acetate.

6. The intermediate as claimed in claim 1, characterised in that the weight ratio of retigabine to surface stabiliser is 1:1 to 1:10.

7. The intermediate as claimed in claim 1, characterised in that the glass transition temperature (Tg) of the intermediate is higher than 20° C.

8. The intermediate as claimed in claim 1, characterised in that it additionally comprises a crystallisation inhibitor based on an inorganic salt, an organic acid, a polymer with a weight-average molecular weight of more than 500,000 g/mol or mixtures thereof.

9. The intermediate as claimed in claim 8, wherein the crystallisation inhibitor is citric acid, ammonium chloride, Povidone K 90 or mixtures thereof.

10. A process for preparing an intermediate as claimed in claim 1, comprising the steps of
(a1) dissolving retigabine and the surface stabiliser in a solvent or mixture of solvents, and
(b1) spraying the solution from step (a1) onto a substrate core.

11. A process for preparing an intermediate as claimed in claim 1, comprising the steps of
(a2) dissolving retigabine and the surface stabiliser in a solvent or mixture of solvents, and
(b2) spray-drying the solution from step (a2).

12. A process for preparing an intermediate as claimed in claim 1, comprising the steps of
(a3) mixing retigabine and surface stabiliser, and
(b3) melt-processing the mixture, the melt-processing conditions being selected such that there is a transition from crystalline to non-crystalline retigabine.

13. A process for preparing an intermediate as claimed in claim 1, comprising the steps of
(a4) dissolving retigabine and the surface stabiliser in a solvent or mixture of solvents, and
(b4) freeze-drying the solution from step (a4).

14. An intermediate obtainable by a process as claimed in claim 10.

15. A pharmaceutical formulation comprising non-crystalline retigabine in the form of an intermediate as claimed in claim 1.

16. The pharmaceutical formulation as claimed in claim 15, comprising
(i) 1 to 50% by weight amorphous retigabine, and
(ii) 5 to 25% by weight disintegrants, based on the total weight of the dosage form.

17. The pharmaceutical formulation as claimed in claim 16, characterised in that it is an alkaline disintegrant.

18. The pharmaceutical formulation as claimed in claim 15 in the form of capsules or tablets, wherein the tablets are produced by means of dry granulation.

19. A method of identifying a pharmaceutical excipient which is suitable as a surface stabiliser for solid, non-crystalline retigabine, comprising the steps of:
a) providing a pharmaceutical excipient which is present in a solid aggregate state at 25° C.,
b) twice in succession, heating up the solid excipient by DSC, and
c) selecting the excipient as suitable if a glass transition point of 25° C. to 100° C. can be seen in the second DSC heating curve.

20. An intermediate comprising solid, non-crystalline retigabine and a pharmaceutical excipient, wherein the excipient has been identified with a method as claimed in claim 19.
